(19) United States
(12) Patent Application Publication
(10) Pub. No.: US 2023/0158021 A1
(43) Pub. Date: May 25, 2023

(54) SARS-CoV-2 Antiviral Pharmaceutical Composition and Application Thereof

(71) Applicant: (LIMITED LIABILITY COMPANY "CROMIS" (LLC "CROMIS")), Moscow (RU)

(72) Inventors: Alexandre Vasilievich Ivachtchenko, Hallandale, FL (US); Andrey Alexandrovich Ivashchenko, Moscow (RU); Nikolay Filippovich Savchuk, Rancho Santa Fe, CA (US); Alena Alexandrovna Ivachtchenko, Hallandale, FL (US); Dmitrii Vladimirovich Kravchenko, Khimki (RU); Tagir Alievich Sitdekov, Moscow (RU); Aleksei Petrovich Il`in, Khimki (RU); Natalia Aleksandrovna Papazova, Liubertsy (RU)

(21) Appl. No.: 17/922,581

(22) PCT Filed: Feb. 4, 2021
(86) PCT No.: PCT/RU2021/000046

§ 371 Date: Oct. 31, 2022

(30) Foreign Application Priority Data
May 7, 2020 (RU) 2020116521
Sep. 2, 2020 (RU) 2020129082

(51) Int. Cl. A61K 31/4965; A61K 9/48; A61K 9/28

(52) U.S. Cl. CPC A61K 31/4965; A61K 9/4858; A61K 9/28

(57) ABSTRACT

This invention relates to a novel anti-RNA, including anti-SARS-CoV-2, viral pharmaceutical composition of Avipiravir in tablets or capsules containing 40-48 wt % of micronized favipiravir, the other ingredients being excipients.

The drug Antiprovir for the prevention and treatment of coronavirus disease COVID-19 is a pharmaceutical composition in the form of coated tablets containing 200 mg, 300 mg, 400 mg, or 600 mg of micronized favipiravir (44.1% by weight-45.6% by weight) with a particle size of 40-211 microns, the other ingredients being excipients.

### FIELD OF INVENTION

This invention relates to a novel anti-SARS-CoV-2 viral pharmaceutical composition for the therapy of RNA virus diseases, including for the prevention and treatment of SARS-CoV mediated COVID-19 virus infection. The sudden 2019 outbreak of a new coronavirus, later named SARS-CoV-2, in Wuhan, China, which quickly became a global pandemic, marked the third introduction of virulent Coronavirus into human society, affecting not only the healthcare system but also the global economy. Effective approaches to vaccination, prevention, and treatment of SARS-CoV-2 (COVID-19) and epidemiological control are still lacking.

### BACKGROUND OF THE INVENTION

In this regard, an intensive worldwide search is underway for vaccines and therapeutic agents to prevent and treat SARS-CoV-2 (COVID-19). One practical approach as a rapid response to an emerging pandemic is to repurpose existing therapeutic agents previously intended for other viral infections, since most of these agents have already been tested for their safety.

The anti-influenza medication Favipiravir (FVP), also known as T-705 and Avigan, was patented in 1999 by the Japanese company Toyama Chemical Co [RU 2224520]. FVP is used in Japan to treat influenza, including the highly pathogenic A H5N1 strain of avian influenza [R. W. Sidwell at al. Antimicrob. Agents Chemother. 2007, 51(3): 845-851].

FVP shows antiviral activity against many other RNA viruses, such as arenaviruses, bunyaviruses, and filoviruses, which are known to cause fatal hemorrhagic fever [Y. Furuta et al. Review Favipiravir (T-705), a broad spectrum inhibitor of viral RNA polymerase. Proc. Jpn. Acad., Ser. B 93, 2017, 449-463].

FPV was successfully tested for the treatment of a progressive infection in mice caused by the Ebola virus [L. Oestereich et al. Ebola virus infection with T-705 (favipiravir) in a small animal model. Antiviral Research 2014, 105, 17-21.] and is an encouraging drug candidate, but it has not been yet approved by the WHO.

Ebola, also known as Ebola virus disease (EVD) and Ebola hemorrhagic fever (EHF) or simply Ebola fever, is a viral hemorrhagic fever in humans and other primates caused by

Ebolaviruses. The disease has a high risk of death, killing 25% to 90% of those infected, with an average of about 50%. EVD outbreaks occur intermittently in tropical regions of sub-Saharan Africa. Between 1976 and 2012, according to the World Health Organization, there were 24 outbreaks of Ebola resulting in a total of 2,387 cases and 1,590 deaths. The largest outbreak to date was in West Africa, which occurred between December 2013 and January 2016, with 28,646 cases and 11,323 deaths. An Ebola vaccine was approved in the United States in December 2019. As of 2019, there was no approved treatment for Ebola [https://en.wikipedia.org/wiki/Ebolavirusdisease].

In February 2020, FPV was successfully tested in an initial randomized trial in China as an antiviral therapy for SARS-CoV-2 (COVID-19) coronavirus. FPV received short-term approval in China on Feb. 16, 2020 as an effective antiviral against COVID-19 for five years. It is currently produced in China under the name Favilavir [https://de.wikipedia.org/wiki/Favipiravir].

FPV sold in Japan under the brand name Avigan (Avigan Tablet 200 mg, Toyama Chemical Co.) and in China under the brand name Favilavir (Favilavir Tablet 200 mg, Zhejiang Hisun Pharm.) is an antiviral drug used in Japan to treat flu. It is being developed and produced by Toyama Chemical Co. (Fujifilm Group) and was approved for medical use in Japan in 2014 [Shiraki K., Daikoku T. Favipiravir, an anti-influenza drug against life-threatening RNA virus infections. Pharmacology & Therapeutics 2020, 107512. doi:10.1016/j.pharmthera.2020.107512].

In 2016, Fujifilm licensed FPV to Chinese pharmaceutical company Zhejiang Hisun [E. J. Lane (Jun. 22, 2016). “Fujifilm in Avigan API license with Zhejiang Hisun Pharmaceuticals”. Retrieved Apr. 20, 2020.]. In 2019, FPV became a generic. In 2010, PCT international application JP 2010/054191 (Mar. 12, 2010) [WO 2010/104170 (Sep. 16, 2010)] was published, under which RU 2527766 C2 was issued for “Tablets and granular powders containing 6-fluoro-3-hydroxy-2-pyrazinecarboxamide.” On Mar. 15, 2020, FPV was approved in China for the treatment of coronovirus. [Yangfei Z. “Potential coronavirus drug approved for marketing”. Chinadaily.com.cn. Retrieved 2020-03-21].

As of Apr. 30, 2020, COVID-19 coronavirus affected 210 countries and territories, with a total of 3,308,233 cases of SARS-CoV-2 infected people, of which 1,042,819 people recovered and 234,105 people died [https://www.worldometers.info/coronavirus/? utm_campaign=homeAdvegas1?].

Given that SARS-CoV-2 poses a serious threat to the world's public health and economy, it seems appropriate to search for novel effective anti-coronavirus agents. The known 200 mg Avigan coated tablets of Toyama Chemical Co., LTV Toyama, are protected by patent RU 2527766, according to which the FPV content of the tablet is 50-95%. However, in all of the 19 examples presented, the FPV content of the tablets varies in the range of 70-80%.

Disclosure of Invention

The subject matter of the present invention is a novel anti-SARS-CoV-2 virus pharmaceutical composition containing 40-48 wt % of micronized favipiravir with a particle size of 40-211 μm, the other ingredients being excipients.

Preferably, the composition contains 44.1-45.6 wt % of micronized favipiravir.

The excipients of the composition may be selected from fillers, disintegrants, binders, glidants, and lubricants. The proposed composition can be used primarily for the prevention and treatment of COVID-19.

Another subject of the present invention is the use of said composition for preparing a dosage form in tablets or capsules.

Preferably, the composition contains 44.1-45.6 wt % of micronized favipiravir, 5.5÷6.0 wt % of croscarmellose sodium, 4.8÷5.0 wt % of povidone, 0.6÷0.8 wt % of magnesium stearate, 0.5÷0.7 wt % of colloidal silicon dioxide, and the rest is microcrystalline cellulose.

Preferred is Avifavir in the form of coated tablets or capsules containing less than 45% by weight of micronized FPV with a particle size of less than 60 microns, and including 200 mg, 300 mg, 400 mg, or 600 mg of FPV with improved solubility.

Avifavir in the form of coated tablets may include 43.3 wt % of micronized FPV, 42.1% microcrystalline cellulose, 5.8% croscarmellose sodium, 4.9% povidone, 0.7% magnesium stearate, 0.6% colloidal silicon dioxide, and 2.6% film coating.

As can be seen from Table 1, the decrease in FPV particle size in the coated tablet pharmaceutical composition leads to a significant improvement in its main parameter—the time of FPV release from the tablet in various media. Indeed, the release time of FPV from tablet 3 containing the lowest FPV particle size significantly exceeds the time of FPV release from tablets 1 and 2, wherein the particle size exceeds 60 μm.

It should be noted that the reduced FPV content also results in a lower time of FPV release from the pharmaceutical composition in coated tablets. Thus, the release percentage for FPV in coated tablets in a solution with a pH of 4.5 according to Examples 2 and 3 (Table 1) from patent RU 2527766 containing 79% and 86% FPV, respectively, is 93.5% and 86.7% in 15 minutes. In contrast, tablet 3 of the present invention containing 43.3% FPV releases a higher percentage of FPV (98.1%, Table 1) and does it three times faster (in 5 minutes).

A similar pattern is observed when comparing the release times (95.1%-99.7% in 15 minutes) of FPV in coated tablets containing 77.5% FPV in a solution with a pH of 4.5 according to Examples 4÷10 (Table 2) from patent RU 2527766. At the same time, tablet 3 according to the present invention containing 43.3% FPV releases 100% (Table 1) FPV in 10 minutes.

Note that tablets 3 of the present invention release FPV in solutions with pH=1.2 and pH=6.8 rapidly and practically quantitatively (Table 1).

TABLE 1
Avifavir coated tablet formulations and FPV release rates as a function
of FPV particle size, acidity of solutions, and mixing time.

| | Avifavir coated tablet formulations, mg | | |
|---|---|---|---|
| Composition | 1* | 2** | 3* |
| FPV | 200.00 | 200.00 | 200.00 |
| | (42.28%) | (45.8%) | (43.3%) |
| FPV microcrystal size, μm | 67.1 | 211.0 | 40-50 |
| Filler: microcrystalline | 213.14 | — | 194.65 |
| cellulose MCC 102 | | | |
| Filler: Prosolv SMCC 90 | — | 194.8 | |
| Disintegrant: croscarmellose | — | 21 | 27.0 |
| sodium | | | |
| Glidant: Aerosil 200 | 2.275 | 1.26 | |
| Disintegrant: L-HPC LH-21 | 22.75 | — | |
| Binder: Povidone K30 | 13.65 | — | 22.5 |
| Lubricant: magnesium stearate | 3.185 | 2.94 | 3.15 |
| Colloidal silica (USP/NF, | | | 2.7 |
| Ph. Eur.) | | | |
| Coating: Opadry 85F38183 | 18.2 | 16.8 | 12.0 |
| yellow | | | |
| Total | 473.20 | 436.80 | 462.0 |

| Medium*** | Time | Percent of FPV release | | |
|---|---|---|---|---|
| pH 1.2 | 5 min | 36.6 | 62.1 | 89.6 |
| | 10 min | 73.8 | 76.0 | 100.5 |
| | 15 min | 81.6 | 82.7 | 101.6 |
| pH 4.5 | 5 min | 50.0 | 78.3 | 98.1 |
| | 10 min | 83.7 | 83.5 | 100.6 |
| | 15 min | 89.3 | 85.3 | 101.2 |
| pH 6.8 | 5 min | 45.7 | 70.2 | 100.0 |
| | 10 min | 83.2 | 75.0 | 101.8 |
| | 15 min | 90.1 | 77.5 | 102.4 |

*micronized FPV and wet granulation,
**direct pressing.
***The study was carried out on a paddle stirrer at 75 rpm and 37 ± 0.5° C. The dissolution medium with pH 1.2: 0.2% sodium chloride solution in 0.1M hydrochloric acid solution. The dissolution medium with pH 4.5: sodium acetate buffer solution (quality control medium). The dissolution medium with pH 6.8: phosphate buffer solution.

In addition, by reducing the FPV content in the pharmaceutical composition of Avifavir it became possible to significantly improve the technological properties of the granulate (pharmaceutical composition) such as flowability and compressibility thus ensuring high pressing productivity and high content uniformity in each unit dosage form.

The efficient method for manufacturing Avifavir tablets and their specific composition make it possible to reduce the time costs of the production process. The high productivity of the tableting and film coating stages is provided by the excellent processability of the granulate and a polyvinyl alcohol-based film coat enabling the use of a more concentrated suspension with a solid content of up to 20%.

### BEST EMBODIMENT OF INVENTION

This invention is illustrated by, but not limited to, the following examples.

Example 1. Preparation of an Avifavir pharmaceutical composition in capsules, each containing 200 mg (45%) of FPV. Two hundred grams of micronized FPV with a microcrystal size of 40-50 μm and 250 grams of lactose powder are thoroughly mixed. The resulting powdered mixture is packed in 450 mg portions into suitably sized gelatin capsules, each containing 200 mg (44.4%) of FPV.

Example 2. Preparation of an Avifavir pharmaceutical composition in coated tablets, each containing 200 mg, 300 mg, 400 mg, or 600 mg FPV (formulation 3 in Table 1). All raw materials are weighed, and magnesium stearate is sifted for dusting. Micronized FPV with a microcrystalline size of 40-50 μm (200 g), microcrystalline cellulose MCC 102 (194.65 g), croscarmellose sodium (27.0 g), and 2.7 g of colloidal silicon dioxide (USP/NF, Ph.Eur.) are sequentially loaded into a granulator mixer, and the components are mixed until the mixture is homogeneous. With constant stirring of the mixture, a previously prepared 6% solution of povidone K30 (22.5 g) is loaded into the granulator mixer, in full, until the end point of granulation is reached. The wet granulate is calibrated through a 2.0 mm sieve. The calibrated wet granulate is dried to the specified residual humidity. The dried granulate is calibrated through a 0.5 mm sieve with setting the optimal fractional composition. The resulting granulate is powdered in a mixer with pre-sifted magnesium stearate (3.15 g). The powdered mixture is divided into three parts and tableted on a rotary tablet press. The resulting core tablets with a mass of ≈450 mg, ≈675 mg, or ≈1350 mg containing 200 mg, 300 mg, 400 mg, or 600 mg of FPV, respectively, each having a hardness of 60 N, an abrasion of no more than 5%, and a disintegration of no more than 3 min are transferred to the coating stage. The process of film coating (Opadry 85F38183 yellow) is carried out in a tablet coater until the target weight of an Avifavir tablet weighing 462 mg, 693 mg, or 1386 mg, respectively, is reached.

Similarly, pharmaceutical compositions of Avifavir in coated tablets are obtained according to formulations 1 and 2 (Table 1).

Example 3. The kinetics of dissolution of coated Avifavir tablets containing 200 mg FPV in three buffers.

The study of the kinetics of dissolution of Avipiravir in coated tablets containing 200 mg FPV was carried out in accordance with the guidelines for the examination of medicines [Guidelines for the Examination of Medicines. Volume 1. Moscow: Grif and K., 2013, 328 pp., Chapter 7.5; Guidelines for the Examination of Medicines. Volume 3, Moscow: POLYGRAPHPLUS, 2014, 344 pp., Chapter 11.] in three buffer media with pH=1.2, 4.5, and 6.8 to model the main areas of the gastrointestinal tract wherein the release and absorption of the active ingredient occurs. The following media were used in the study: 0.2% sodium chloride solution in 0.1 M hydrochloric acid with pH 1.2, sodium acetate buffer solution with pH 4.5 (quality control medium), and phosphate buffer solution with pH 6.8 all prepared in accordance with the EP.7.0.5.17.1 «Recommendations on Dissolution Testings». Time sampling points were selected to provide a reliable description of the dissolution profile with a gradual increase and subsequent attainment of complete release (at least 85% of the active ingredient) or plateau. To study dissolution kinetics, the following time sampling points were selected: 5 min, 10 min, 15 min, 20 min, and 30 min. To obtain statistically reliable results for each drug, the test was carried out on 12 dosage form units. The quantitative content of FPV released into the dissolution medium was determined by HPLC. The calculations took into account the change in the volume of the dissolution medium.

Dissolution kinetics was studied using a DT828 Tablet/Capsule Dissolution Tester (Erweka, Germany). Quantification was performed using liquid chromatographs: Agilent 1260 (Agilent Technologies, USA) with OpenLab ChemStation software and LC-20A Prominence (Shimadzu, Japan) with LabSolutions software. The laboratory scales MV210A (SartoGosm, Russia), Acculab VIC-210d2 (Acculab, Sartorius Group, USA), and Quintix 64-1ORU (Sartorius Group, Germany) as well as the pH-meter SEVEN MULTI (Mettler Toledo, Switzerland) were used as supporting equipment. Statistical processing of the experimental results was performed using Microsoft Office Excel 2007. We used volumetric glassware of classes “A” (volumetric flasks of 50, 500, 1000 ml), “AS” (1- and 5-ml analytical pipettes), and “B” (100-, 250- and 1000-ml volumetric cylinders).

The test was conducted in accordance with the requirements of SPRF XIV, GPM 1.4.2.0014.15 “Dissolution for Solid Dosage Forms”. [SPRF XIV, Vol. 2, 2018, 2164 pp., GPM.1.4.2.0014.15 Dissolution for Solid Dosage Forms], “Guidelines for the examination of medicinal products”. [Guidelines for the Examination of Medicinal Products. Vol. 1. Moscow: Grif & K., 2013, 328 pp., Chapter 7.5. Guidelines for the Examination of Medicinal Products. Volume 3. Moscow: POLIGRAFPLUS, 2014, 344 pp., Chapter 11.] as well as in accordance with the recommendations of the scientific and practical guidelines for the pharmaceutical industry “Dissolution Test in the Development and Registration of Medical Products” edited by I. E. Shokhin [Test in the Development and Registration of Medical Products. Scientific and Practical Guidlines for the Pharmaceutical Industry, Ed. by Shokhin I. E. Moscow: Pero Publ., 2015, 320 pp.]

Test conditions: apparatus type—paddle stirrer; temperature −37±0.5° C.; medium volume −900 ml; rotation speed −75 rpm; time sampling points −5, 10, 15, 20, and 30 min. The results of the evaluation of FPV release from three formulations of Avipiravir tablets as a function of FPV particle size, solution acidity, and stirring time are presented in Table 1.

### INDUSTRIAL APPLICABILITY

The invention can be used in medicine and veterinary medicine.

1. An anti-SARS-CoV-2 viral pharmaceutical composition containing 40-48 wt % of micronized favipiravir with a particle size of 40-211 μm, the other ingredients being excipients.

2. The composition according to claim 1 containing 44.1-45.6 wt % of micronized favipiravir.

3. The composition according to any one of claims 1-2 containing excipients selected from a range of fillers, disintegrants, binders, glidants, and lubricants.

4. The composition according to any one of claims 1-3 for the prevention and treatment of COVID-19.

5. The use of the anti-SARS-CoV-2 viral pharmaceutical composition according to any one of claims 1-4 to obtain a dosage form in tablets or capsules.
